SEQUENCE LISTING (9 sequences)

SEQ ID NO: 1 - protein, 213 aa, Artificial Sequence
  C. albicans ATCC 10231
MKKTALIVVDLQEDFLPPNGSLAIKNGRSVIPKINQLLPSQDNHSKFDWSLIVATQDWHPPNHTSFASQHENVAPFTEIEFIHPEKKLDPKTNQPIVMNQIVWPDHCVQGTKGAQLEPLFANQFEKLTKQDDNNTAPCKIVKKGYLPDREYYSCFQDCWGLHHTELIDLLHEYDIENVVFVGLAYDFCVLRSAIDSAKNGFKTFVLKNYCESV

SEQ ID NO: 2 - protein, 186 aa, Artificial Sequence
  S. areus ATCC 6538
MTHRALLVVDYSYDFIADDGLLTCGKPGQNIEDFIVSRINDFNYYQDHIFFLMDLHYLHDIHHPESKLFPPHNIVDTSGRELYGKVGKLYETIKAQPNVHFIDKTRYDSFFGTPLDSLLRERSINQVEIVGVCTDICVLHTAISAYNLGYKISVPAEGVASFNQKGHEWALAHFKNSLGAEVEQHV

SEQ ID NO: 3 - protein, 183 aa, Artificial Sequence
  B. subtilis ATCC 6633
MKKALICIDYTNDFVASDGKLTCGEPGRMIEEAIVNLTEEFITNGDYVVLAVDSHDEGDQYHPETRLFPPHNIKGTDGKDLYGKLLPLYQKHEHEPNVYYMEKTRYSAFAGTDLELKLRERQIDELHLAGVCTDICVLHTAVDAYNKGFRIVVHKQAVASFNQEGHTWALSHFANSIGAQVAE

SEQ ID NO: 4 - protein, 218 aa, Artificial Sequence
  P. aeruginosa ATCC 9027
MNRPKIASFDVDAQKGFTPLCPDELPVPGGDAIGAALNLLASRAALRLGSKDAHSPKAAWVVESAAQMLRPLAYPDADLTWVAHCVPGTPGFELLDELPRPIDYDFFVWKGVEPDLHPYGACYHDLAERRSTGVIEYLRQNGVSHVLVGGLALDYCVKNTALQLRRAGFEVLLYLPACRAIAEDTAQRACDEMRDAGVILCADLERLDSALAGADKEK

SEQ ID NO: 5 - protein, 224 aa, Artificial Sequence
  A. brasiliensis ATCC 16404
MKAALIIVDMQEDFCPQNGSLAVQEARDLAPTINTLLATPGFTLRIASQDSHPPNHISFAPNHPPPNNLPFEHFIEMSNPAAGKETETKPQRLWPVHCVEGTKGAEIIPEIDTKNIDLYVRKGMDARVEMYSAFADAFGNLDAEVNRSSVDVHLKGALEDKGITDVFCVGVAGDYCVKFTAIDAARAGLRSYFVEDAVRCVDPGQGWEEAKRELKEAGVVIVRS

SEQ ID NO: 6 - protein, 186 aa, Artificial Sequence
  C. sporogenes ATCC 15579
MFKSILIIDCQNDFISGSLACDNGELAVKNIINLLNINKDLKVFYSLDWHSENNKSFKINGGIWPVHCVADRFGAKLEKSFYKELQHKNHSPNNKKILFSKGKNDYIEEYSAYEGENILGNKINEVLDKDVIVCGIASEFCVKETVEDLIRNGFNVELYVDGVGYVNKKEHEKTLNNLRKQGAKFI

SEQ ID NO: 7 - protein, 213 aa, Artificial Sequence
  PNCA_ECOLI
MPPRALLLVDLQNDFCAGGALAVPEGDSTVDVANRLIDWCQSRGEAVIASQDWHPANHGSFASQHGVEPYTPGQLDGLPQTFWPDHCVQNSEGAQLHPLLHQKAIAAVFHKGENPLVDSYSAFFDNGRRQKTSLDDWLRDHEIDELIVMGLATDYCVKFTVLDALQLGYKVNVITDGCRGVNIQPQDSAHAFMEMSAAGATLYTLADWEETQG

SEQ ID NO: 8 - protein, 186 aa, Artificial Sequence
  PNCA_MYCTU
MRALIIVDVQNDFCEGGSLAVTGGAALARAISDYLAEAADYHHVVATKDFHIDPGDHFSGTPDYSSSWPPHCVSGTPGADFHPSLDTSAIEAVFYKGAYTGAYSGFEGVDENGTPLLNWLRQRGVDEVDVVGIATDHCVRQTAEDAVRNGLATRVLVDLTAGVSADTTVAALEEMRTASVELVCSS

SEQ ID NO: 9 - protein, 216 aa, Artificial Sequence
  PNC1_YEAST
MKTLIVVDMQNDFISPLGSLTVPKGEELINPISDLMQDADRDWHRIVVTRDWHPSRHISFAKNHKDKEPYSTYTYHSPRPGDDSTQEGILWPVHCVKNTWGSQLVDQIMDQVVTKHIKIVDKGFLTDREYYSAFHDIWNFHKTDMNKYLEKHHTDEVYIVGVALEYCVKATAISAAELGYKTTVLLDYTRPISDDPEVINKVKEELKAHNINVVDK

(19) United States
(12) Patent Application Publication
(10) Pub. No.: US 2024/0200119 A1
(43) Pub. Date: Jun. 20, 2024

(54) METHOD OF DETECTING BACTERIAL/FUNGAL CONTAMINATION

(71) Applicants: Nanyang Technological University, Singapore (SG); Massachusetts Institute of Technology, Cambridge, MA (US); National University of Singapore, Singapore (SG)

(72) Inventors: Jiayi HUANG, Singapore (SG); Scott Alan RICE, Singapore (SG); Stacy L. SPRINGS, Cambridge, MA (US); Yie Hou LEE, Singapore (SG)

(21) Appl. No.: 18/288,401

(22) PCT Filed: Apr. 18, 2022
(86) PCT No.: PCT/SG2022/050227

§ 371 Date: Oct. 26, 2023

(30) Foreign Application Priority Data
Apr. 30, 2021 (SG) 10202104519X

(51) Int. Cl. C12Q 1/22; C12Q 1/34

(52) U.S. Cl. CPC C12Q 1/22; C12Q 1/34

(57) ABSTRACT

The present invention relates to a method of detecting the presence of a bacterial and/or fungal cell in a sample through detecting the presence of nicotinamidase activity or nicotinamidase, wherein the presence of nicotinamidase activity or nicotinamidase indicates the presence of a bacterial and/or fungal cell in the sample. The invention also relates to a method for monitoring bacterial and/or fungal cell contamination in a cell of tissue culture comprising detecting the presence of nicotinamidase activity or nicotinamidase in the cell or tissue culture.

### FIELD

The present disclosure relates to the field of molecular biology. In particular, the specification teaches a method of detecting the presence of a bacterial and/or fungal cell in a sample.

### BACKGROUND

Sterility biotesting is a key requirement for cell and gene therapy product (CTP) manufacturing and release testing. Currently, culture-based methods remain the gold standard to ensure sterility as regulated by FDA, USP and European Pharmacopoeia. However, culture-based methods can take up to 14 days for bacteria and fungi detection, which is incompatible with the short shelf life of CTPs. Hence, the industry has focused on developing alternative test methods that are rapid and that show equivalent performance as compendial reference methods.

Blood culture systems such as BACTEC (Becton Dickinson) and BacT/ALERT (BioMerieux) have been widely used as alternative testing methods. Although these platforms show great performance by providing automated continuous monitoring and objective detection of microbial growth in CTPs, they still require incubation of the product in aerobic and anaerobic enriched medium for up to 7 days. In addition, based on CO₂ measurements, the BacT/ALERT® system might be influenced by the metabolically active cells contained in the product. Thus, an evaluation system needs to distinguish between microbial growth and metabolism of the cell matrix.

Accordingly, there is a need to overcome, or at least to alleviate, one or more of the above-mentioned problems.

### SUMMARY

Disclosed herein is a method for detecting the presence of a bacterial and/or fungal cell in a sample, the method comprising detecting the presence of nicotinamidase activity or nicotinamidase, wherein the presence of nicotinamidase activity or nicotinamidase indicates the presence of a bacterial and/or fungal cell in the sample.

Disclosed herein is a method for detecting bacterial and/or fungal cell contamination in a sample, the method comprising detecting the presence of nicotinamidase activity or nicotinamidase, wherein the presence of nicotinamidase activity or nicotinamidase indicates bacterial or fungal cell contamination in the sample.

Disclosed herein is a method for assessing the sterility of a sample, the method comprising detecting the presence of nicotinamidase activity or nicotinamidase in the sample, wherein the lack of nicotinamidase activity or nicotinamidase as compared to a reference indicates that the sample is sterile, sterilized or free from bacterial and/or fungal cell contamination.

Disclosed herein is a method for monitoring bacterial and/or fungal cell contamination in a cell or tissue culture, the method comprising detecting the presence of nicotinamidase activity or nicotinamidase in the cell or tissue culture, wherein the presence of nicotinamidase activity or nicotinamidase indicates bacterial and/or fungal cell contamination in the cell or tissue culture.

Disclosed herein is a kit for detecting the presence of a bacterial and/or fungal cell in a sample, the method comprising determining the levels of nicotinic acid and nicotinamide in the sample, wherein a change in the ratio of nicotinic acid to nicotinamide levels as compared to a reference indicates the presence of the bacterial and/or fungal cell in the sample.

### BRIEF DESCRIPTION OF THE DRAWINGS

Some embodiments of the present invention will now be described by way of non-limiting example only, with reference to the accompanying drawings in which:

FIG. 1. Schematic for a method as defined herein.

FIG. 2. Experimental flowchart. Three groups of samples were generated in this contamination experiment. Group 1: Uncontaminated MSCs, which are not contaminated by bacteria; Group 2: MSCs contaminated by Staphylococcus epidermidis, Escherichia coli, Klebsiella pneumoniae, Pseudomonas aeruginosa, Staphylococcus aureus and Acinetobacter baumannii; Group 3: the same six bacteria grown in blank DMEM medium as Group 2. All samples were prepared in triplicate.

FIG. 3. PLS-DA score plot based on cell culture supernatant in positive mode. Blue group, bacteria free, MSC culture medium; Green group, culture medium of contaminated MSCs by six bacteria; Red group, Bacteria in culture medium. All the samples were prepared in triplicate and used in PLS-DA.

FIG. 4. Extracted chromatograms and mass spectra of metabolite m/z 123.0366 and metabolite m/z 122.0571 in all samples. A. Representative examples of EIC chromatograms obtained from the uncontaminated MSC culture medium and contaminated MSC culture medium. B. MS/MS identification of nicotinic acid: (a) The product ion spectrum of metabolite m/z 123.0366 in positive ion mode; (b) MS/MS spectrum of nicotinic acid in Metlin database; ion matches are highlighted. C. MS/MS spectrum of nicotinamide in Metlin database; (i) MS/MS spectrum of metabolite m/z 122.0571 in positive mode; (b) MS/MS spectrum of nicotinamide in Metlin database; ion matches are highlighted.

FIG. 5. Analysis of pathway and related enzymes involved in nicotinic acid production. A. Mechanism of nicotinic acid production. Bacteria and fungi possess the pncA gene which encodes for nicotinamidase that converts nicotinamide to nicotinic acid as illustrated by the tested six organisms, while mammals do not possess this gene. B. Multiple sequence alignment between putative nicotinamidases from the USP<71> defined five organisms, C. sporogenes ATCC 15579 and reported nicotinamidases. Residues involved in cis-peptide bond (●) and in the metal ion binding (▪) are shown. The residues forming the active site are indicated by stars (★). Reported nicotinamidases are PNCA_ECOLI for E. coli (Uniprot P21369), PNCA_MYCTU for Mycobacterium tuberculosis (Uninprot: I6XD65) and PNC1_YEAST for Saccharomyces cerevisiae (Uniprot: P53184). QA-Quinolinate; NaMN-Nicotinate mononucleotide; NMN-Nicotinamide mononucleotide.

FIG. 6. Nicotinic acid (NA) to nicotinamide (NAM) ratio for uncontaminated and contaminated MSC culture medium with six different bacteria. With an inoculation condition of 1×10⁴ CFU/mL for 24 h, the ratio of NA to NAM in infected MSC culture medium was 72 to 11,590 times higher than the uncontaminated MSC culture medium. Blank DMEM medium was used as negative control. *P<0.05 compared to blank medium group using One-way ANOVA.

FIG. 7. NA to NAM ratio for uncontaminated and contaminated MSC culture medium by four USP<71> defined organisms. With an inoculation condition of 10⁴ CFU/mL for 24 h, the ratio of NA to NAM in contaminated MSC culture medium was 100 to 8,600 times higher than the uncontaminated MSC culture medium. *p<0.05 compared to blank medium group using t-test.

FIG. 8. NA to NAM ratio for uncontaminated and contaminated MSC culture medium by A. brasiliensis ATCC 16404. With an inoculation condition of 10 CFU/mL for 72 h, the ratio of NA to NAM in contaminated MSC culture medium was 200 times higher than the uncontaminated MSC culture medium. *p<0.05 compared to uncontaminated MSCs group using t-test.

FIG. 9. NA to NAM ratio for uncontaminated and contaminated T cell culture medium with six different bacteria. A. Representative examples of EIC chromatograms obtained from the uncontaminated culture medium and contaminated culture medium. B. NA to NAM ratio of uncontaminated and contaminated T-cell culture medium by six different bacteria. With an inoculation condition of 1×10⁴ CFU/mL for 24 h, the ratio of NA to NAM in contaminated T cell culture medium was 4,500 to 15,000 times higher than the uncontaminated T cell culture medium. Blank RPMI medium was used as negative control. *p<0.05 compared to blank medium group using One-way ANOVA.

FIG. 10. NA to NAM ratio for uncontaminated and contaminated T cell culture medium by four USP<71> defined organisms. In uncontaminated 10⁴, 10⁵ and 10⁶ activated T-cells, no significant increase of NA/NAM ratio was observed. In T-cells inoculated with 10⁴ CFU/mL of USP <71> organisms, NA/NAM ratio increased ranging from 15 to 14,285 times. *p<0.05 compared to blank medium group using t-test.

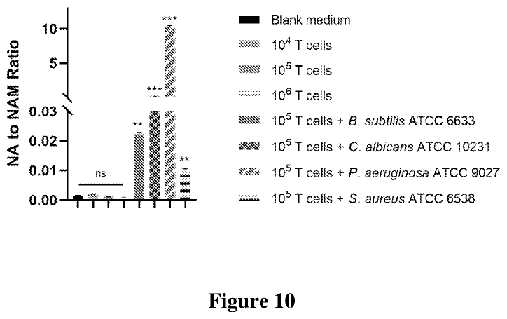

FIG. 11. NA to NAM ratio for uncontaminated and contaminated MSC culture medium with different bacteria. A. With an inoculation condition of 1.4×10⁴ CFU/mL of E. coli, the ratio of NA to NAM in contaminated MSC culture medium became significantly higher than the uncontaminated MSC culture medium after 6 h. B. With an inoculation condition of 18 CFU/mL of E. coli, the ratio of NA to NAM in contaminated MSC culture medium became significantly higher than the uncontaminated MSC culture medium after 18 h. C. With an inoculation condition of 20 CFU/mL of B. subtilis, the ratio of NA to NAM in contaminated MSC culture medium became significantly higher than the uncontaminated MSC culture medium after 12 h. D. With an inoculation condition of 10 CFU/mL of C. albicans, the ratio of NA to NAM in contaminated MSC culture medium became significantly higher than the uncontaminated MSC culture medium after 24 h. *p<0.05 using t-test.

FIG. 12. Inhibition of the production of NA in bacterial contaminated cell culture medium. A. Effect of 1 mM pyrazinecarbonitrile on NA to NAM ratio to MSCs contaminated by ˜200 CFU/mL of E. coli. B. Representative chromatograms obtained from the uncontaminated culture medium and contaminated culture medium using QQQ LC-MS. The ratio of NA to NAM in contaminated MSC culture medium was significantly lower than the MSC culture added with 1 mM pyrazinecarbonitrile.

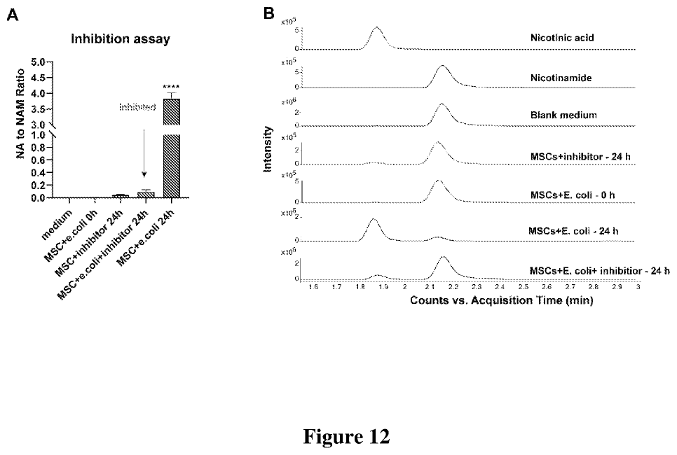

FIG. 13. The structures of nicotinic acid and nicotinamide. A. nicotinic acid. B. nicotinamide.

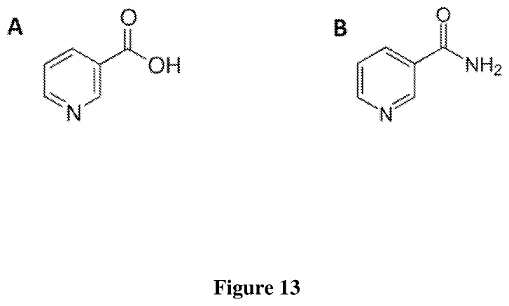

FIG. 14. Schematic diagram illustrating the at-line detection method from a culture flask using the aseptic sampler and optical flow cell.

FIG. 15. Normalized absorbance spectra of culture media spiked with known concentrations of NA and the PLS regression plots for the test data measured in both cuvette and flow cell

### DETAILED DESCRIPTION

Disclosed herein is a method for detecting the presence of a bacterial and/or fungal cell in a sample, the method comprising detecting the presence of nicotinamidase activity or nicotinamidase, wherein the presence of nicotinamidase activity or nicotinamidase indicates the presence of a bacterial and/or fungal cell in the sample.

The absence of nicotinamidase activity or nicotinamidase may indicate the absence of a bacterial and/or fungal cell in the sample.

Without being bound by theory, the inventors have found that it is possible to detect broad-based bacterial contamination in cell and gene therapy products (CTPs) with faster speed, smaller product volume and without the need to perform cell or target labelling or cell lysis. The test can be performed without the need of transferring CTPs to certain medium for enrichment and incubation. In addition, other types of detection techniques including ELISA, antibody/nanobody/recombinant protein-based lateral flow assay and optical detection can be developed based on this invention.

The terms “detecting”, “determining”, “measuring”, “evaluating”, “assessing” and “assaying” are used interchangeably herein to refer to any form of measurement, and include determining if an element is present or not. These terms include both quantitative and/or qualitative determinations. Assessing may be relative or absolute.

In one embodiment, the detection of nicotinamidase activity comprises detecting the conversion of nicotinamide to nicotinic acid. The conversion of nicotinamide to nicotinic acid indicates the presence of a bacterial and/or fungal cell due to the presence of nicotinamidase in the bacterial and/or fungal cell.

In one embodiment, the detection of nicotinamidase activity comprises determining the level of nicotinic acid in a sample, wherein a change in the level of nicotinic acid level as compared to a reference indicates the presence of a bacterial and/or fungal cell in the sample. The change in the level of nicotinic acid level may be an increase in the level of nicotinic acid as compared to a reference.

As used herein, the term “increase” or “increased” with reference to a compound, such as nicotinic acid, may refer to a statistically significant and measurable increase in the compound as compared to a reference. The increase may be an increase of at least about 10%, or an increase of at least about 20%, or an increase of at least about 30%, or an increase of at least about 40%, or an increase of at least about 50%.

In one embodiment, the detection of nicotinamidase activity comprises determining the levels of nicotinic acid and nicotinamide in a sample, wherein a change in the ratio of nicotinic acid level to nicotinamide level as compared to a reference indicates the presence of a bacterial and/or fungal cell in the sample.

In one embodiment, an increase in the ratio of nicotinic acid level to nicotinamide level as compared to a reference indicates the presence of a bacterial and/or fungal cell in a sample.

As used herein, the term “increase” or “increased” with reference to a ratio of nicotinic acid level to nicotinamide level may refer to a statistically significant and measurable increase in the ratio as compared to a reference. The increase may be an increase of at least about 10%, or an increase of at least about 20%, or an increase of at least about 30%, or an increase of at least about 40%, or an increase of at least about 50%.

In one embodiment, a decrease in the ratio of nicotinamide level to nicotinic acid level as compared to a reference indicates the presence of a bacterial and/or fungal cell in a sample.

As used herein, the term “decrease” or “decreased” with reference to a ratio of nicotinamide level to nicotinic acid level refers to a statistically significant and measurable decrease in the ratio as compared to a reference. The decrease may be a decrease of at least about 10%, or a decrease of at least about 20%, or a decrease of at least about 30%, or a decrease of at least about 40%, or a decrease of at least about 50%.

In one embodiment, the levels of nicotinic acid and nicotinamide are detected using liquid chromatography-mass spectrometry (LC-MS) analysis, UV or Raman spectroscopy, paper-based diagnostic method using protein binders.

In one embodiment, the detection of nicotinamidase comprises detecting the nicotinamidase gene or gene expression product, wherein the presence of nicotinamidase gene or gene expression product indicates the presence of a bacterial and/or fungal cell in a sample.

In one embodiment, the nicotinamidase gene or gene expression product is detected by PCR or a protein-capture based technique. Such techniques are well-known in the art.

The methods of the invention may be practiced using any sample suspected of containing a bacterial and/or fungal cell. In one embodiment, the sample is a sample from a therapeutic product (such as a biopharmaceutical product). The sample may be a sample obtained from a biopharmaceutical manufacturing process for a therapeutic product. The therapeutic product may, for example, be a cell, gene, protein, antibody or vaccine therapeutic product.

In one embodiment, the therapeutic product is a cell and gene therapeutic product (CTP). The CTP may include cellular immunotherapies, cancer vaccines, and other types of both autologous and allogeneic cells for certain therapeutic indications, including hematopoetic stem cells and adult and embryonic stem cells. CTPs can include T cell, CART-T cell (e.g. Yescarta™ (axicabtagene ciloleucel), Kymriah™ (tisagenlecleucel)), or NK cell therapies. The CTP may be a gene therapeutic product that seeks to modify or manipulate the expression of a gene or to alter the biological properties of living cells for therapeutic use.

In one embodiment, the sample is a cell or tissue culture supernatant sample. In one embodiment, the sample is a mammalian cell culture supernatant sample. In one embodiment, the sample is a cell culture supernatant sample from a cell and gene therapy product (CTP).

A sample can be a biological sample which refers to the fact that it is derived or obtained from a living organism. The organism can be in vivo (e.g. a whole organism) or can be in vitro (e.g., cells or organs grown in culture). A “biological sample” also refers to a cell or population of cells or a quantity of tissue or fluid from a subject. Most often, a sample has been removed from a subject, but the term “biological sample” can also refer to cells or tissue analyzed in vivo, i.e., without removal from the subject. Often, a “biological sample” will contain cells from a subject, but the term can also refer to non-cellular biological material, such as non-cellular fractions of blood, saliva, or urine. The biological sample may be from a resection, bronchoscopic biopsy, or core needle biopsy of a primary, secondary or metastatic tumor, or a cellblock from pleural fluid. In addition, fine needle aspirate biological samples are also useful. Biological samples also include explants and primary and/or transformed cell cultures derived from patient tissues. A biological sample can be provided by removing a sample of cells from subject, but can also be accomplished by using previously isolated cells or cellular extracts (e.g. isolated by another person, at another time, and/or for another purpose). Archival tissues, such as those having treatment or outcome history may also be used. Biological samples include, but are not limited to, tissue biopsies, scrapes (e.g. buccal scrapes), whole blood, plasma, serum, urine, saliva, cell culture, or cerebrospinal fluid. In one embodiment, the biological sample is obtained in a clinical setting such as in the clinic or hospital.

In one embodiment, the sample may be a non-clinical sample from foodstuff, beverages, pharmaceuticals, cosmetics, water (e.g., drinking water, non-potable water, and waste water), seawater ballasts, air, soil, sewage, plant material (e.g., seeds, leaves, stems, roots, flowers, fruit), shampoos and other consumer products. The cell culture supernatant may be one that contains nicotinamide in the cell culture medium.

The method may comprise the addition of nicotinamide to the sample.

The “reference” as referred to herein may be one or more samples that does not have bacterial or fungal contamination. The reference may also be a pre-determined value or an average value. In one embodiment, the method as defined herein comprises the step of comparing to a reference. The method may comprise, for example, comparing the ratio of nicotinic acid to nicotinamide in the sample as compared to the ratio of nicotinic acid to nicotinamide in a reference. In one embodiment, the reference is a cell culture sample that does not have bacterial or fungal contamination. The method may also comprise comparing the ratio of nicotinic acid to nicotinamide in the sample to a threshold value. The threshold value may be, for example, 0.05, 0.1, 0.2, 0.3, 0.3, 0.4, 0.5, 0.6, 0.7, 0.8, 0.9, 1 or more.

In one embodiment, the bacterial or fungal cell comprises or expresses a nicotinamidase enzyme. The bacterial or fungal cell may express the nicotinamidase enzyme. In one embodiment, the bacterial or fungal cell is one that possesses the pncA gene which encodes for nicotinamidase.

In one embodiment, the bacterial cell is a Gram positive bacterial cell selected from the group consisting of Bacillus subtilis, Staphylococcus capitus, Actinomyces turicensis, Bacillus cereus, Staphylococcus warneri, Streptococcus agalactiae, Streptococcus pyrogenes, Propionibacterium acnes, Clostridium sporogenes, and Corynebacterium amycolatum. In one embodiment, the bacterial cell is a Gram negative bacterial cell selected from the group consisting of Acinetobacter lwoffii and Pseudomonas fluorescens.

In one embodiment, the bacterial or fungal cell is selected from the group consisting of Staphylococcus epidermidis, Escherichia coli, Klebsiella pneumoniae, Pseudomonas aeruginosa, Staphylococcus aureus, Acinetobacter baumannii, Bacillus subtilis, Clostridium sporogenes, Candida albicans, Aspergillus brasiliensis, Mycoplasma fermentans, Mycoplasma orale and Mycoplasma synoviae.

In one embodiment, the bacterial or fungal cell is selected from the group consisting of Staphylococcus epidermidis, Escherichia coli, Klebsiella pneumoniae, Pseudomonas aeruginosa, Staphylococcus aureus, Acinetobacter baumannii, Bacillus subtilis, Clostridium sporogenes, Candida albicans, and Aspergillus brasiliensis. In one embodiment, the bacterial or fungal cell is selected from the group consisting of Mycoplasma fermentans, Mycoplasma orale and Mycoplasma synoviae.

Disclosed herein is a method for detecting bacterial and/or fungal cell contamination in a sample, the method comprising detecting the presence of nicotinamidase activity or nicotinamidase, wherein the presence of nicotinamidase activity or nicotinamidase indicates bacterial and/or fungal cell contamination in a sample.

Disclosed herein is a method for monitoring bacterial and/or fungal cell contamination in a cell or tissue culture, the method comprising detecting the presence of nicotinamidase activity or nicotinamidase in the cell or tissue culture, wherein the presence of nicotinamidase activity or nicotinamidase indicates bacterial and/or fungal cell contamination in the cell or tissue culture.

The method may comprise obtaining a sample, such as a supernatant sample, from the cell or tissue culture.

Disclosed herein is a method for assessing the sterility of a sample, the method comprising detecting the presence of nicotinamidase activity or nicotinamidase in the sample, wherein the lack of nicotinamidase activity or nicotinamidase as compared to a reference indicates that the sample is sterile, sterilized or free from bacterial or fungal cell contamination.

Disclosed herein is a sample comprising a bacterial and/or fungal cell, wherein the sample comprises nicotinamidase activity or nicotinamidase. In one embodiment, the nicotinamidase activity or nicotinamidase indicates the presence of the bacterial and/or fungal cell.

Disclosed herein is a sample which is contaminated with a bacterial and/or fungal cell, wherein the sample comprises nicotinamidase activity or nicotinamidase. In one embodiment, the presence of nicotinamidase activity or nicotinamidase indicates contamination by the bacterial and/or fungal cell.

Disclosed herein is a kit for detecting the presence of a bacterial and/or fungal cell in a sample, the method comprising determining the levels of nicotinic acid and nicotinamide in the sample, wherein a change in the ratio of nicotinic acid to nicotinamide levels as compared to a reference indicates the presence of the bacterial and/or fungal cell in the sample.

Disclosed herein is a method for detecting a bacterial and/or fungal infection in a subject, the method comprising detecting the presence of nicotinamidase activity or nicotinamidase, wherein the presence of nicotinamidase activity or nicotinamidase indicates the presence of a bacterial and/or fungal infection in the subject.

As used herein, the term “subject” includes any human or non-human animal. In one embodiment, the subject is a human. The term “non-human animal” includes all vertebrates, e.g., mammals and non-mammals, such as non-human primates, sheep, dog, cow, chickens, amphibians, reptiles, etc.

Disclosed herein is a method of treating a bacterial and/or fungal infection in a subject, the method comprising a) detecting the presence of nicotinamidase activity or nicotinamidase, wherein the presence of nicotinamidase activity or nicotinamidase indicates the presence of a bacterial and/or fungal infection in the subject; and b) treating the bacterial and/or fungal infection in the subject.

The term “treating” or “treatment” as used herein may refer to (1) preventing or delaying the appearance of one or more symptoms of the disorder; (2) inhibiting the development of the disorder or one or more symptoms of the disorder; (3) relieving the disorder, i.e., causing regression of the disorder or at least one or more symptoms of the disorder; and/or (4) causing a decrease in the severity of one or more symptoms of the disorder.

Methods of treating a bacterial and/or fungal infection are well known in the art. For example a subject may be administered an antimicrobial agent (such as an antibiotic) or an anti-fungal agent.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Certain embodiments of the invention will now be described with reference to the following examples which are intended for the purpose of illustration only and are not intended to limit the scope of the generality hereinbefore described.

### EXAMPLES

### Methods

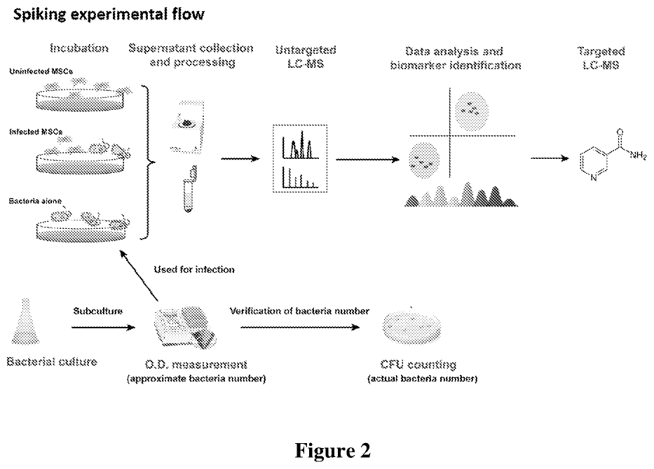

For a spiking experiment, CTPs such as mesenchymal stem cells are cultured at 37° C. with 5% CO2 in Dulbecco's Modified Eagle Medium (DMEM) supplemented with 10% fetal bovine serum (FBS) and with 1% (v/v) antibiotics (100 U/mL penicillin and 100 ng/mL streptomycin). The cells are grown in a T175 flask until they reached a 70% to 80% confluence. CTPs such as T cells were seeded in fresh AIM V medium supplemented with 2% AB human serum and IL2 (100 IU/mL). To activate T cells, 25 μL of ImmunoCult™ Human CD3/CD28 T cell activator was added to 1 mL of 1 million of cell suspension and incubated at 37° C. with 5% CO2. After 3 days of activation, a viable cell count was performed and the viable cell density was adjusted every 2-3 days by adding fresh complete AIM V+2% AB human serum+IL2 (100 IU/ml) to the cell suspension. Bacteria are plated onto Luria broth (LB) agar plates and cultivated at 37° C. overnight. After the incubation, a single colony of each bacteria species is isolated and inoculated into 5 mL of LB broth (Sigma-Aldrich, St. Louis, U.S.A.) for overnight culture at 37° C. Next morning, the overnight culture is subjected to optical density (O.D.) measurement at 600 nm by a ULTROSPEC® 10 cell density meter (Biochrom Ltd., Cambridge, UK). Subsequently, the bacterial suspension was diluted to OD600 nm=0.075 and further subcultured at 37° C. for the bacteria to reach log phase. When the OD600 nm reach the range between 0.6 and 0.8, the bacterial culture is used for the infection experiments. Viable counting of bacteria suspensions in different experiments showed that an OD600 nm of 0.1 corresponds to approximately 1×10⁷ CFU/mL. Cells are cultivated as mentioned above and were trypsinized from culture flask and suspended in the culture medium without antibiotics. 1×10⁵ cells per well of cells were seeded in 6-well plates and incubated overnight at 37° C. with 5% CO₂. Serial 10-fold dilution of bacterial suspensions at log phase are performed in PBS. Bacterial dilution corresponding approximately to 10, 100, 1000 and 1×10⁴ CFU/mL bacteria per well are inoculated, each individual strain separately, into the cells which have been cultured overnight. In parallel, same amount of each bacterial strains are inoculated into the blank medium separately. All treatments are performed in triplicate. The detailed grouping information can be found in FIG. 2. The observation of cellular morphology is performed after infection experiments using phase-contrast microscopy.

For a CTP product release testing, cell culture supernatant is collected from the end product. For in-process monitoring, cell culture supernatant is collected every 1 h.

For every experiment, 100 μL cell culture from each well was collected and filtered through a Nanosep® centrifugal devices with Omega™ 10K molecular weight cut-off membrane (Pall Corporation, New York, USA) by centrifugation at centrifuged at 8,000×g for 5 min. The filtrates were collected in autosampler vials for liquid chromatography-mass spectrometry (LC-MS) analysis. LC-MS analysis is performed with Agilent 1290 ultrahigh pressure liquid chromatography system (Waldbronn, Germany) coupled to an electrospray ionization with iFunnel Technology on a triple quadrupole mass spectrometer. Chromatographic separation is achieved by using ACQUITY UPLC HSS T3 (2.1×100 mm, 1.8 μm; Waters, Milford, MA, USA) column with a Waters ACQUITY HSS T3 1.8 μM VANGUARD guard column. Mobile phase consists of (A) 0.1% formic acid in water and (B) 0.1% formic acid in methanol. The initial condition is set at 0% B for 3 min. A 2 min linear gradient to 95% B is applied and then is held for 5 min. Then, it is returned to starting conditions over 1 min. The column is kept at 50° C.

The auto-sampler is cooled at 4° C. and an injection volume of 0.5 μL with a flow rate of 0.3 mL/min is used. Electrospray ionization is performed in positive ion mode with the following source parameters: drying gas temperature 200° C. with a flow of 14 L/min, nebulizer gas pressure 30 psi, sheath gas temperature 400° C. with a flow of 11 L/min, capillary voltage 3,000 V and nozzle voltage 800 V. Multiple reaction monitoring (MRM) mode is used to monitor the transitions m/z 124.04>79.90 and m/z 123.06>80.00 for nicotinic acid and nicotinamide, respectively.

Raw spectrometric data are analyzed by MassHunter Qualitative Analysis software (Agilent Technologies, CA, USA). The molecular features of the peaks are obtained using the Molecular Feature Extraction algorithm based on the analysis of their retention time, chromatographic peak intensity and accurate mass. A Mass Hunter Mass Profiler Professional software (Agilent Technologies, CA, USA) is used to visualize and analyze the features. For further processing, the features are filtered by criteria that with an intensity ≥5,000 counts and found in at least 50% of the samples at the same sampling time point signal. To align the retention time and m/z values, a tolerance window of 0.15 min and 2 mDa was used.

### Example 1

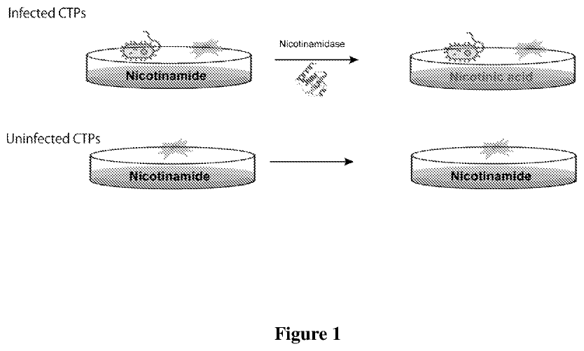

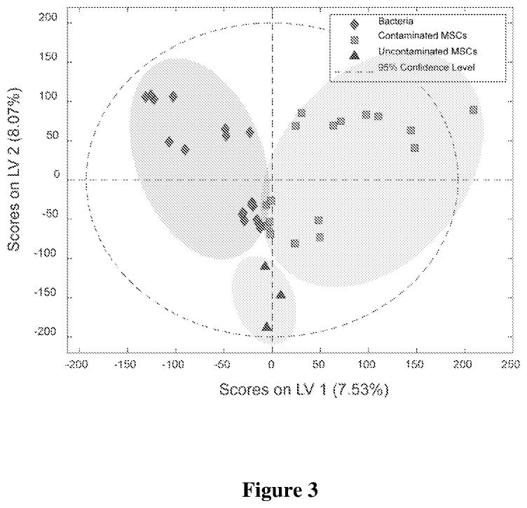

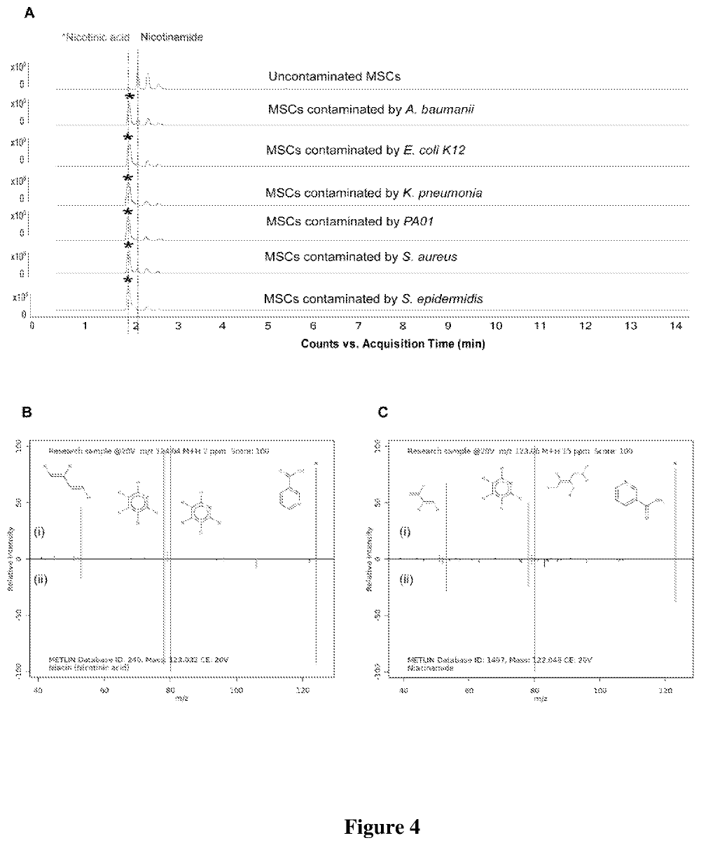

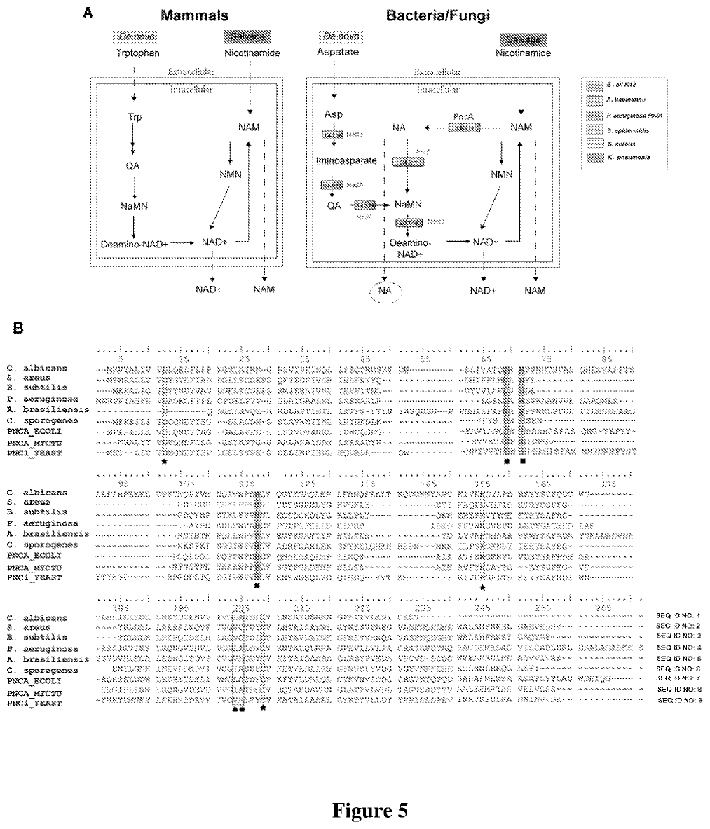

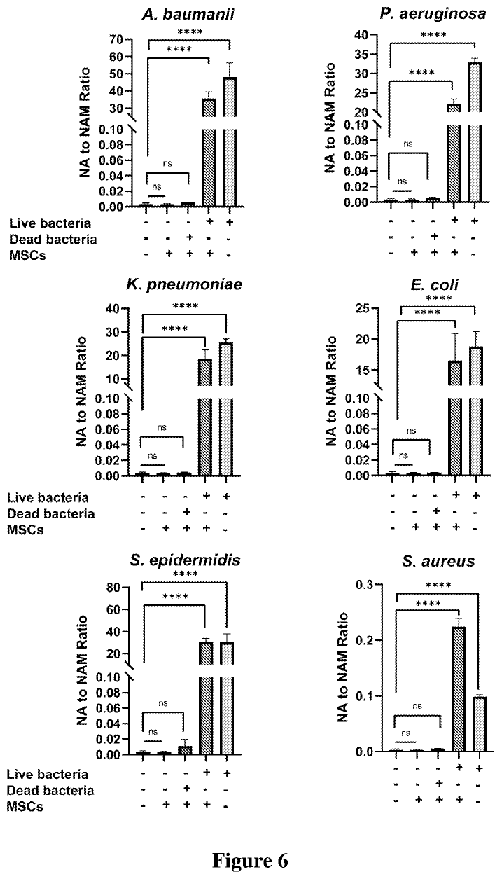

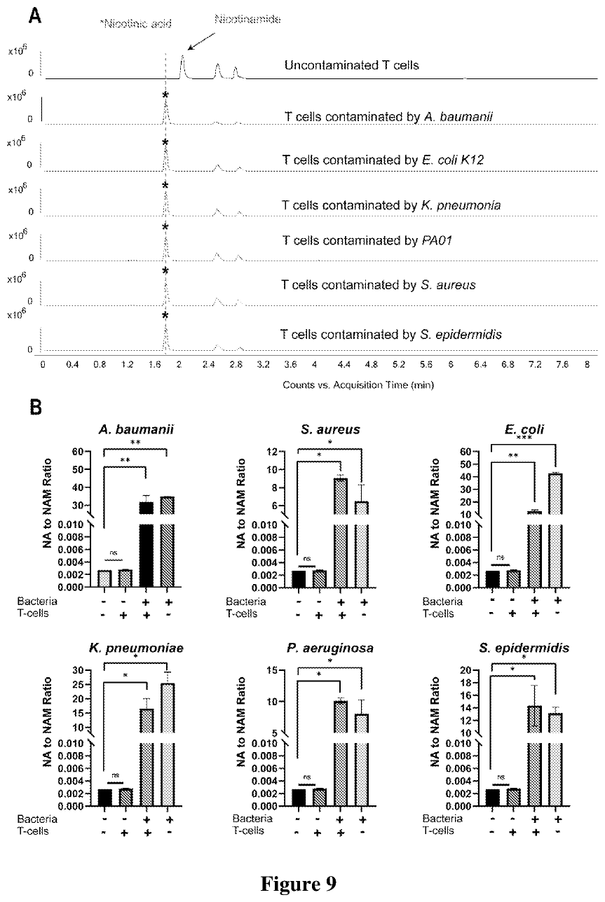

The present disclosure identifies the use of a unique broad-based bacteria metabolite nicotinic acid (NA) and its precursor nicotinamide (NAM) for indicating cell therapy product (CTP) bacterial contamination using LC-MS analysis (see FIGS. 1 to 4). NA is produced from the medium containing (NAM) by bacterial nicotinamidase, which is not present in mammalian cells. PubSeed database and literatures revealed at least 9582 of bacteria organisms and 31 fungi species (due to the low numbers of fungal genome being characterized) possess the gene PncA which encodes nicotinamidase (FIG. 5). The ratio of NA to NAM in culture supernatant increased significantly after cell infection and hence served as a sterility critical quality attribute for assessing bacterial contamination of CTPs. Six different bacteria from five genus (Staphylococcus epidermidis (ATCC 12228), Escherichia coli K12 (ATCC 25404), Klebsiella pneumoniae (ATCC KP1), Pseudomonas aeruginosa PA01 (ATCC BAA-47), Staphylococcus aureus (ATCC 25923) and Acinetobacter baumannii were employed in the study using mesenchymal stem cells (MSCs) and activated T cells (FIGS. 6 and 9). With an contamination condition of 1×10⁴ CFU/mL for 24 h, the ratio of NA to NAM was 72 to 15,000 times higher than the uncontaminated cells. In addition, E. coli was used as an example to validate the results.

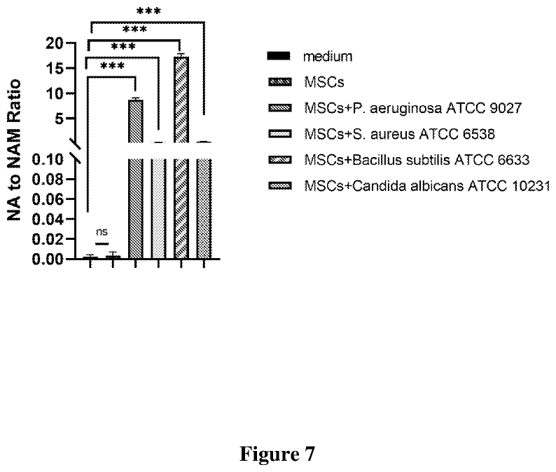

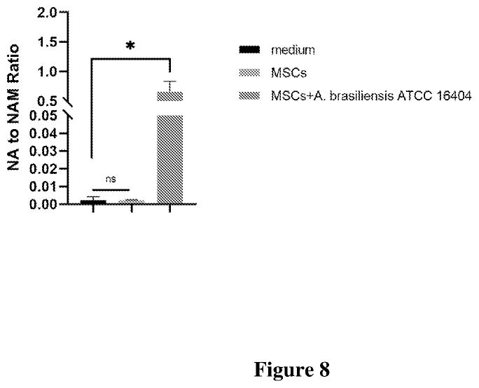

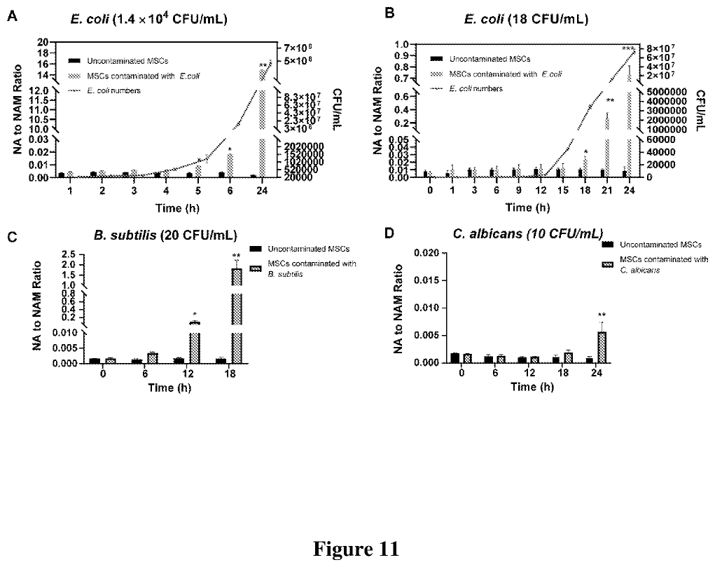

Low inoculum numbers (˜20 CFU/mL) and high inoculum numbers (˜1×10⁴ CFU/mL) of E. coli were used to infect the MSCs, showing that significant increase in NA to NAM ratio were observed after 6 h and 18 h infection time (FIG. 11). Furthermore, for method suitability testing, five of the USP<71> defined organisms Staphylococcus aureus ATCC 6538, Pseudomonas aeruginosa ATCC 9027, Bacillus subtilis ATCC 6633, Candida albicans ATCC 10231 and Aspergillus brasiliensis ATCC 16404 have been challenged and their NA to NAM ratio were evaluated (FIGS. 7 and 8).

### Testing of Mycoplasma Species

A genome database search was also performed for identifying the presence of the pncA gene which encodes nicotinamidase in Mycoplasma. Among the Mycoplasma species that are specified in USP <63>, Mycoplasma fermentans ATCC 19989, Mycoplasma orale ATCC 23714 and Mycoplasma synoviae ATCC 25204 possess the pncA gene encoding nicotinamidase. The remaining strains, which include Acholeoplasma laidlawii ATCC 23206, Mycoplasma gallisepticum ATCC 19610, Mycoplasma hyorhinis ATCC 17981 and Mycoplasma pneumoniae ATCC 15531, do not appear to possess pncA gene and hence will not be detected using the methods as defined herein. Therefore, the method is useful for detecting some of the Mycoplasma contaminations in cell therapy products.

Detection Limit and Comparison with Compendial Sterility Test

To compare the performance of NA to NAM ratio detection method to the gold standard method, the same amount of E. coli K12 was inoculated with MSCs and 1 mL of the cell culture were collected at 0 h and inoculated into 9 mL of soybean casein digest at 20-25° C. for visual observation as USP <71> sterility test suggests. USP <71> sterility test showed a visible turbidity of 18 CFU/mL of E. coli after 24 h, while a significant increase of the NA/NAM ratio was observed after 18 h. Similarly, visible turbidity was observed for an inoculation of B. subtilis with 20 CFU/mL after 24 h, while a significant increase of the NA/NAM ratio was observed after 12 h (FIG. 11C). Also, the time to detection for 10 CFU/mL of C. albicans was 48 h using USP <71> sterility testing method, while the time to detection was 24 h based on the NA/NAM ratio increase (FIG. 11D). In conclusion, the method based on the NA/NAM ratio performed better than the gold standard USP <71> sterility test method in terms of time to detection. The study also demonstrated that the method could achieve a detection limit of 10 CFU, which is comparable with the compendial sterility test and other alternative microbiological methods. For the automated blood culture systems such as Bactec (Becton, Dickinson) and BacT/Alert (bioMérieux), they took approximately 15.5 h, 16.0 h and 27.7 h to detect 10 CFU of E. coli, B. subtilis and C. albicans at 32.5° C., respectively. In comparison, our method showed faster or equivalent detection speed for low inoculum compared to the automated blood culture systems depending on different bacterial species. The results show that the NA to NAM ratio in cell culture medium can be used to indicate the early stages of bacterial contamination.

### Example 2

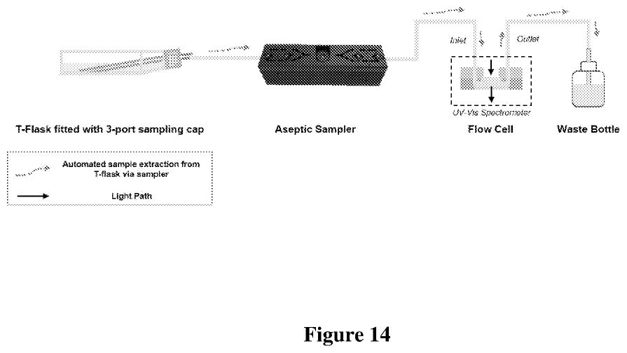

NA is produced when a bacterial enzyme metabolizes a specific compound in the cell culture medium. It has a unique signature in the ultraviolet (UV) wavelength regime, enabling its detection through UV absorbance spectroscopy. This technique is highly reproducible and exhibits high sensitivity even with measurement times. Hence, it is an excellent candidate for real-time monitoring. In this study, a rapid method has been established for quantifying NA in mammalian cell culture medium employing UV absorbance spectroscopy aided by machine learning. In addition, an aseptic instrumentation for at-line monitoring is devised using an automated, aseptic sampler to extract culture supernatant into an optical flow cell for measurements, depicted in FIG. 14. This approach allows for at-line microbial contamination monitoring during cell therapy product manufacturing.

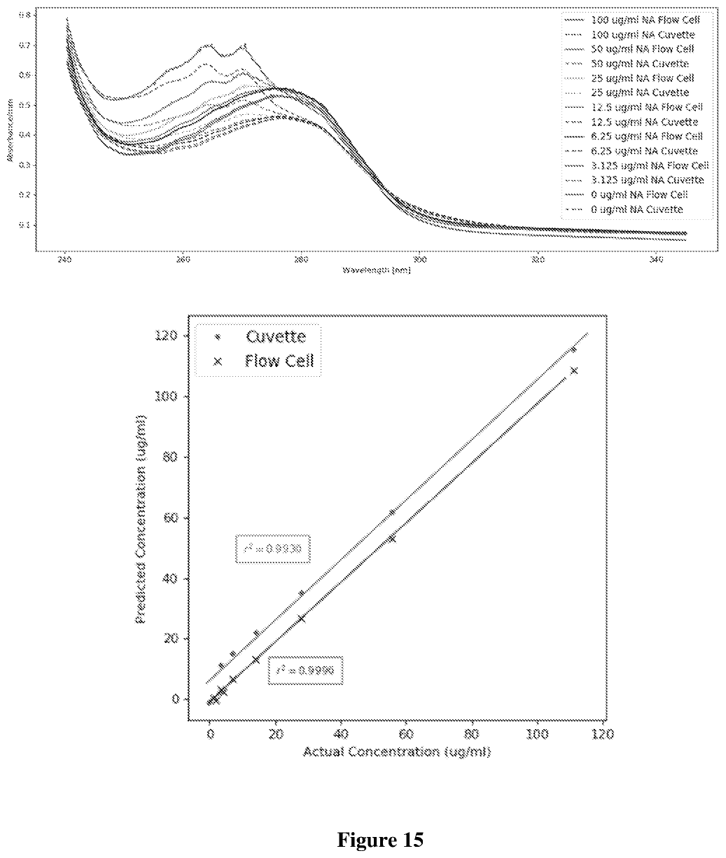

Mammalian culture medium (Dulbecco's Modified Eagle Medium 11885084, from Gibco, supplemented with 10% Fetal Bovine Serum) was spiked with various concentrations of NA and the solution absorbance was measured using a UV-Vis spectrometer (Cary 60, from Agilent). Ten spectra between 240 to 300 nm and the corresponding NA concentrations were introduced into a Partial Least Squares (PLS) regression model as training data. Additional spectra that were not used for the training, obtained from spiked culture media with various NA concentrations were then used for testing to evaluate the model's performance. Preliminary measurements were done in a standard UV quartz cuvette (2 mm path length) and the aseptic optical flow cell (2.8 mm path length). The normalized absorbance spectra for these measurements (FIG. 15) show comparable performance of our optical flow cell for UV spectroscopy. The PLS regression prediction plot from the flow cell measurements demonstrates that this method is sensitive down to 3.125 ug/ml, the lowest NA concentration tested.

In conclusion, the inventors have demonstrated a potential rapid machine learning-aided, at-line bacterial contamination detection method based on UV absorbance spectroscopy, using bespoke aseptic instrumentation. The presence of bacterial contamination can be determined through the detection of NA in the supernatant, without sacrificing cell products. This method is rapid and sensitive for NA concentrations down to 3.125 μg/ml for early contamination detection. The limit of detection can be further improved by increasing the optical path length to increase the signal to noise ratio. Future developments are underway to improve the instrumentation for on-line monitoring capabilities during cell manufacturing.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A method for detecting the presence of a bacterial and/or fungal cell in a sample, the method comprising detecting the presence of nicotinamidase activity or nicotinamidase, wherein the presence of nicotinamidase activity or nicotinamidase indicates the presence of a bacterial and/or fungal cell in the sample.

2. The method of claim 1, wherein detection of nicotinamidase activity comprises determining the levels of nicotinic acid and nicotinamide in a sample, wherein an increase in the ratio of nicotinic acid to nicotinamide levels as compared to a reference indicates the presence of a bacterial and/or fungal cell in the sample.

3. The method of claim 2, wherein an increase in the ratio of nicotinic acid level to nicotinamide level as compared to a reference indicates the presence of a bacterial and/or fungal cell in the sample.

4. The method of claim 3, wherein an increase in the ratio of nicotinic acid level to nicotinamide level as compared to a reference indicates the presence of a live bacterial and/or fungal cell in the sample.

5. The method of any one of claims 2 to 4, wherein the levels of nicotinic acid and nicotinamide are detected using liquid chromatography-mass spectrometry (LC-MS) analysis, UV or Raman Spectroscopy or paper-based diagnostic method using protein binders.

6. The method of claim 1, wherein detecting nicotinamidase comprises detecting the nicotinamidase gene or gene expression product, wherein the presence of nicotinamidase gene or gene expression product indicates the presence of a bacterial and/or fungal cell in the sample.

7. The method of claim 6, wherein the nicotinamidase gene or gene expression product is detected by PCR or a protein-capture based technique.

8. The method of any one of claims 1 to 7, wherein the bacterial and/or fungal cell comprises or expresses a nicotinamidase enzyme.

9. The method of any one of claims 1 to 8, wherein the bacterial and/or fungal cell is one that possess the PncA gene.

10. The method of any one of claims 1 to 9, wherein the sample is a sample from a therapeutic product.

11. The method of any one of claims 1 to 10, wherein the reference is a sample that does not have bacterial and/or fungal contamination.

12. A method for detecting bacterial and/or fungal cell contamination in a sample, the method comprising detecting the presence of nicotinamidase activity or nicotinamidase, wherein the presence of nicotinamidase activity or nicotinamidase indicates bacterial and/or fungal cell contamination in the sample.

13. A method for assessing the sterility of a sample, the method comprising detecting the presence of nicotinamidase activity or nicotinamidase in the sample, wherein the lack of nicotinamidase activity or nicotinamidase as compared to a reference indicates that the sample is sterile, sterilized or free from bacterial and/or fungal cell contamination.

14. A method for monitoring bacterial and/or fungal cell contamination in a cell or tissue culture, the method comprising detecting the presence of nicotinamidase activity or nicotinamidase in the cell or tissue culture, wherein the presence of nicotinamidase activity or nicotinamidase indicates bacterial and/or fungal cell contamination in the cell or tissue culture.

15. The method of claim 14, wherein the method is performed in real-time.

16. A kit for detecting the presence of a bacterial and/or fungal cell in a sample, the kit comprising determining the levels of nicotinic acid and nicotinamide in the sample, wherein a change in the ratio of nicotinic acid to nicotinamide levels as compared to a reference indicates the presence of the bacterial and/or fungal cell in the sample.
